SEQUENCE LISTING (1 sequence)

SEQ ID NO: 1 - protein, 434 aa, Trichoderma reesei
QSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSG

(19) United States
(12) Patent Application Publication
(10) Pub. No.: US 2023/0242955 A1
(43) Pub. Date: Aug. 3, 2023

(54) CHIMERIC CELLOBIOHYDROLASES

(71) Applicant: Alliance for Sustainable Energy, LLC, Golden, CO (US)

(72) Inventors: Michael E. HIMMEL, Littleton, CO (US); Stephen R. DECKER, Berthoud, CO (US); Roman BRUNECKY, Arvada, CO (US); Venkataramanan SUBRAMANIAN, Arvada, CO (US); Brandon Christopher KNOTT, Lakewood, CO (US); Vladimir Vladimirovich LUNIN, Evergreen, CO (US); Todd VANDERWALL, Golden, CO (US)

(21) Appl. No.: 17/899,588

(22) Filed: Aug. 30, 2022

Related U.S. Application Data
(60) Provisional application No. 63/238,747, filed on Aug. 30, 2021.

(51) Int. Cl. C12P 19/02; C12N 9/42

(52) U.S. Cl. CPC C12P 19/02; C12N 9/2437; C12Y 302/01004

(57) ABSTRACT

A range of Cel7 putative cellobiohydrolase genes were identified using genome mining and homologous sequence alignment to Cel7A from Trichoderma reesei. A representative subset of these genes from across a broad diversity of evolutionarily disparate sources were cloned and expressed in T. reesei using a constitutive promotor and a common secretion signal. The purified recombinant enzymes were tested for efficacy on various substrates. The top performers were subjected to structural studies and subsites likely to confer enhanced performance were predicted using homology modeling and comparisons of natural sequence diversity. Once identified, the subsites were genetically introduced individually and combinatorically into the best in class Cel7A backbone we have found to date and then expressed in T. reesei and tested. A triple mutant was determined to have the highest cellulase activity we have measured in a cellobiohydrolase to date.

### CROSS-REFERENCE TO RELATED APPLICATION

This application claims priority under 35 U.S.C. 119(e) to U.S. Provisional Application No. 63/238,747 filed 30 Aug. 2021, the contents of which are incorporated herein by reference.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### SEQUENCE LISTING

The instant application contains a Sequence Listing which has been submitted via the Patent Center and is hereby incorporated by reference in its entirety. The XML copy as filed herewith was originally created on 24 Apr. 2023. The XML copy as filed herewith is named NREL_21-106.xml, is 2,388 bytes in size and is submitted with the instant application.

### BACKGROUND

Many biomass-degrading enzymes under development today are based on fungal cellulase secretomes. The emphasis on fungal cocktails originated from the isolation of the fungus Trichoderma reesei in the late 1940s, which has grown into an important platform for the production of cellulases at extremely high protein titers. In most eukaryotic cellulase systems, and especially in cellulolytic filamentous fungi, Glycoside Hydrolase Family 7 (GH7) cellobiohydrolases (CBHs) are often the main enzymes produced in natural secretomes, likely because these enzymes provide the majority of the hydrolytic activity for cellulose conversion to glucose. GH7 cellulases are particularly important for industrial fungal cellulase cocktails, as the current lignocellulosic biorefineries operating worldwide predominantly use fungal-based cellulase systems. GH7 CBHs have therefore been the focus of many structural and biochemical studies and primary targets for cellulase engineering. Today's formulations employ well studied, primarily naturally occurring enzymes; Nature harbors better examples and these templates can be used to engineer superior enzymes. Cellulosic sugar production requires cheap and highly effective enzymes. By utilizing more efficient enzymes, less enzyme can be used overall, thereby reducing the total cost contribution of enzymes to the overall process. Therefore, we have enhanced the performance of key process hydrolytic enzymes to enable higher sugar yields at lower enzyme loadings. The process may also be completed faster and/or to a greater extent of conversion using enhanced Cell cellobiohydrolases. We wish to reduce overall process uncertainty and cost, as enzymes are ˜10% of minimum fuel selling price (MFSP).

Existing cellulosic enzyme cocktails have been optimized by enzyme production companies for decades, generally through mixing and synergizing different activities to target specific substrates. Enzyme companies have also optimized the production and processing to make these commercially viable; however, there is still a need for further development to be cost effective on biomass conversion.

### SUMMARY

Our “King Chimera” triple mutant cellobiohydrolase was previously demonstrated to be 55% more active than the industry standard Cel7A from Trichoderma reesei when assayed in a minimal enzyme cocktail has been proven to be higher activity when assayed in a complete cellulase background as well. For the first time, we have demonstrated that our improved Cel7A enzymes maintained their relative activity improvements when deployed in the context of a full cellulase system and not just as part of a simplified engineered minimal cellulase cocktail. We used a fully induced broth from our cbh1-delete QM6a strain, AST1116 as our complete cellulase cocktail.

To further test the industrial performance of our improved Cel7A enzymes, we created a commercially relevant cbh1-deleted version of CTec3 by chromatographically removing the endogenous Cel7A cellobiohydrolase and reconstituting the complete commercial CTec3 minus the cellobiohydrolase by combining all non-Cel7A fractions back into a single mix. We are currently testing this as a background cellulase system for the improved variants.

We have cloned the triple mutant “King Chimera” into our artificial multifunctional cellulase analog. Previous work demonstrated that the combined PfCel7A-CBM3-AcCel5A construct had very high activity in comparison to the individual Cel7A and Cel5A proteins. By replacing the PfCel7A cat domain with the King Chimera cat domain, we expect to push this activity even higher.

Other objects, advantages, and novel features of the present invention will become apparent from the following detailed description of the invention when considered in conjunction with the accompanying drawings.

### BRIEF DESCRIPTION OF THE DRAWINGS

FIG. 1 depicts a comparison of enzymes at equal enzyme loading on the same process-relevant treated corn stover substrate. C11-6=Talaromvces aculeatus, CPD14=Aspergillus oryzae, Megatron2=C-10-5/Thielavia terrestris.

FIG. 2 depicts improved Cel7A from diversity studies and extent of glucan conversion of DMR pretreated corn stover using a trinary mixture of E1 endoglucanase, beta-glucosidase, and different cellobiohydrolases. C11-6=Talaromvces aculeatus, HC-10=Penicillium funiculosum, CPD14=Aspergillus oryzae, C-10-5=Thielavia terrestris.

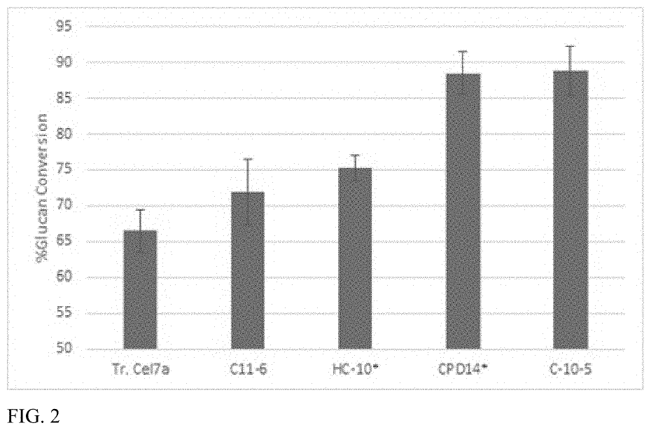

FIG. 3 depicts a multiple alignment of Cel7A amino acid sequences. T. reesei Cel7A (SEQ ID NO: 1) was aligned with the two improved Cel7As namely P. funiculosum and A. oryzae (CPD14), and the newly identified Cel7A sequences from T. terrestris (C10-5) and T. aculeatus (C11-6). The selected mutations are represented by the black box.

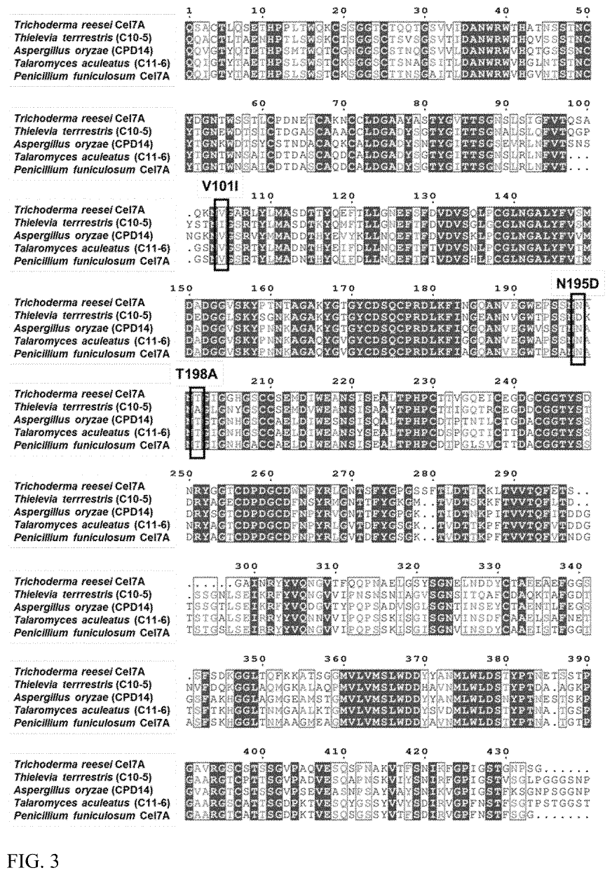

FIG. 4 depicts a model of GH7 CBH “King Chimera”.

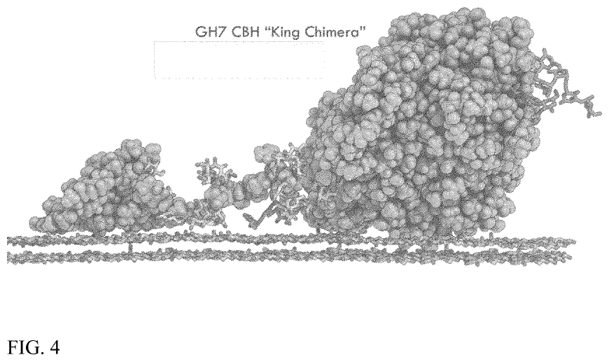

FIG. 5 depicts an addback study of King Chimera Cel7A in a complex cellulase background.

FIG. 6 depicts a plasmid construct for King Chimera variant of the raptor multifunctional cellulase.

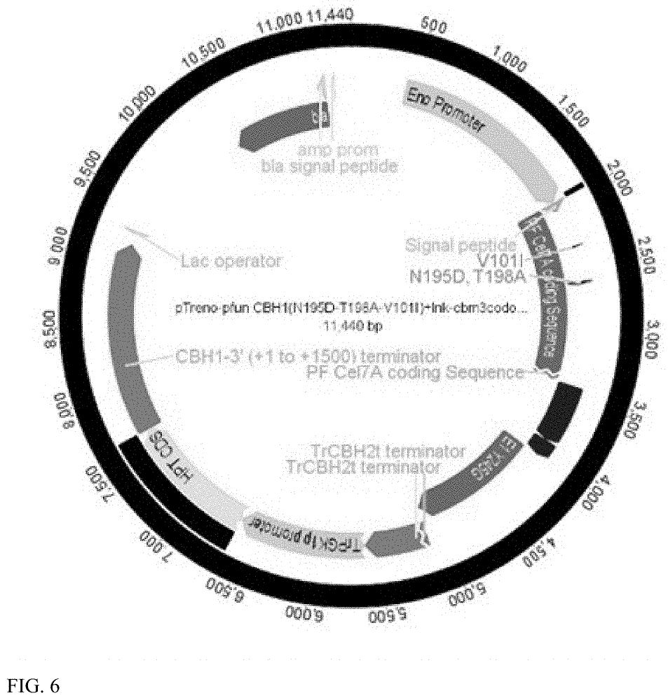

FIG. 7 depicts a linkage analysis of recalcitrant oligomers from enzyme hydrolyzed DMR.

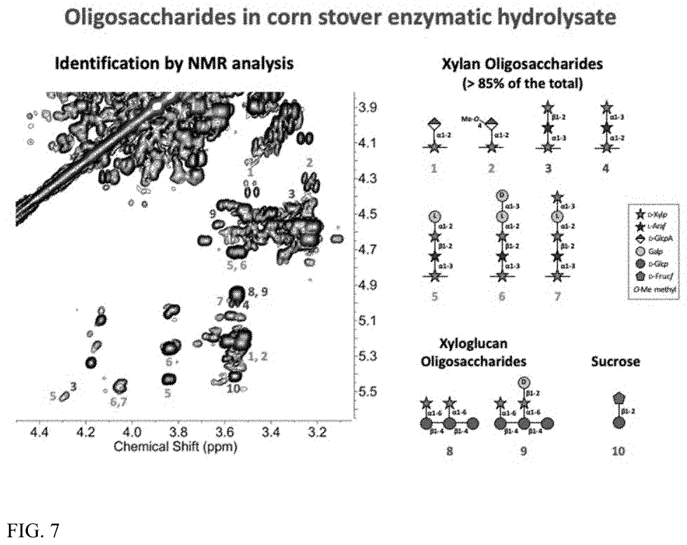

FIG. 8 depicts the results of an addback study comprising Cel7 free background broth that was generated from T. reesei, to this was added either wild type Cel7A or the triple mutant (V101I)(T198A)(N195D).

### DETAILED DESCRIPTION

In most eukaryotic cellulase systems, and especially in cellulolytic filamentous fungi, Glycoside Hydrolase Family 7 (GH7) cellobiohydrolases (CBHs) are often the main enzymes produced in natural secretomes, likely because these enzymes have intrinsically low catalytic rates, yet provide the majority of the hydrolytic activity for cellulose conversion to glucose. The GH7 cellulases are particularly important for industrial fungal cellulase cocktails, as current lignocellulosic biorefineries operating worldwide predominantly use fungal-based cellulase systems. Both endo-acting and exo-acting cellulases are found within the GH7 family, with the exo-acting cellobiohydrolases typically found as the dominant cellulase in fungal secretomes, therefore GH7 CBHs have been the focus of many structural and biochemical studies and primary targets for cellulase engineering. Modern industrial cellulase formulations employ well studied, primarily naturally occurring enzymes which have been developed through activity-based screens and selection. In contrast to quick and simple assays that measure initial rates or biophysical parameters, screens that explore the full extent and capabilities of these enzymes are slow and tedious, so only the tip of the sequence space iceberg for these critical cellulases has been explored.

Cellulosic sugar production requires cheap and highly effective enzymes. By utilizing more efficient enzymes, less enzyme can be used overall, thereby reducing the total cost contribution of enzymes to the overall process. Therefore, we have enhanced the performance of key process hydrolytic enzymes to enable higher sugar yields at lower enzyme loadings.

Disclosed herein are a range of Cel7 putative cellobiohydrolase genes were identified using genome mining and homologous sequence alignment to the amino acid sequence of Cel7A from Trichoderma reesei (SEQ ID NO: 1).

TABLE 1
(SEQ ID NO: 1)
QSACTLQSETHPPLTWQKCSSGGTCTQQTGSVVIDANWRWTHATN
SSTNCYDGNTWSSTLCPDNETCAKNCCLDGAAYASTYGVTTSGNS
LSIGFVTQSAQKNVGARLYLMASDTTYQEFTLLGNEFSFDVDVSQ
LPCGLNGALYFVSMDADGGVSKYPTNTAGAKYGTGYCDSQCPRDL
KFINGQANVEGWEPSSNNANTGIGGHGSCCSEMDIWEANSISEAL
TPHPCTTVGQEICEGDGCGGTYSDNRYGGTCDPDGCDWNPYRLGN
TSFYGPGSSFTLDTTKKLTVVTQFETSGAINRYYVQNGVTFQQPN
AELGSYSGNELNDDYCTAEEAEFGGSSFSDKGGLTQFKKATSGGM
VLVMSLWDDYYANMLWLDSTYPTNETSSTPGAVRGSCSTSSGVPA
QVESQSPNAKVTFSNIKFGPIGSTGNPSG

A representative subset of these genes from across a broad diversity of evolutionarily disparate sources were cloned and expressed in T. reesei using a constitutive promotor and a common secretion signal. The purified recombinant enzymes were tested for efficacy on various substrates. The top performers were subjected to structural studies and subsites likely to confer enhanced performance were predicted using homology modeling and comparisons of natural sequence diversity. Once identified, the subsites were genetically introduced individually and combinatorically into the “best in class” Cel7A backbone we have found to date (in an embodiment, Penicillium funiculosum) and then expressed in T. reesei and tested. A triple mutant was determined to have the highest cellulase activity we have measured in a cellobiohydrolase to date. A 50% more efficient enzyme allows for a lower total enzyme loading to be used enabling major cost savings. We have demonstrated that our chimeric, engineered enzymes can be used at low loadings to achieve superior results beyond naturally available examples.

We identified three probable structural subsites conferring improved performance from natural diversity and then engineered these sites into the PfCel7A chassis generating single, double, and triple chimera mutants many of which showed enhanced performance on DMR solids.

There remains a very large unexplored sequence space for other cellulases such as GH6 exocellulases, GH5/7/9/48 endocellulases, GH1/3 b-glucosidases, and numerous other hemicellulase and accessory enzymes that hold real potential for improving the overall activity of cellulase mixes acting on pretreated biomass.

Our Cel7A engineering efforts continue to generate improvements in activity when assayed against commercially relevant biomass substrate (DMR corn stover). We used process relevant assays (4+ days of hydrolysis) using real-world substrates (DMR corn stover). Our 55% improvement in Cel7A activity through enzyme engineering is the highest we have seen in the published literature for commercially relevant cellobiohydrolases on real substrates achieving industrially relevant high conversion levels. Prior results were obtained on initial activity (5-60 min assay) of the Cel7A variant only on purified cellulose, not biomass. Others have reported up to 4.5-fold higher activity for mutations in the Talaromyces cellulolyticus Cel7A, however, these results were based on initial activity rates (1 h digestions) and are meaningless for the high extent of conversion assays required to evaluate commercial enzymes for use in biomass conversion.

Our application of improved Cel7A variants from the diversity study and activity transfer engineering work has demonstrated increased sugar release from DMR stover, up to 90% conversion of glucan to glucose at 96 hours in a simple trinary enzyme system. This sugar release will be even higher if the engineered Cel7A (King Chimera triple mutant) is incorporated into a commercially engineered cellulase formulation containing additional hemicellulase and cellulase activities.

Our demonstration of retained activities improvements of our engineered Cel7As in a complex cellulase background shows that we can improve commercial cellulases by improving the key Cel7A enzyme.

Design of New Highly Active Chimeric Cellulases

In an embodiment, methods and compositions of matter are disclosed herein to engineer new Cel7A enzymes able to function in a consortium of enzymes able to meet or exceed 90% conversion of glucan and xylan to monomers using 10 mg enzyme/gram loadings of DMR (deacetylated mechanically refined) pretreated corn stover. Our natural diversity screening efforts of Cel7A enzymes resulted in the discovery of multiple native enzymes exhibiting improved characteristics relative to the industry standard Trichoderma reesei enzyme. We posited that if we could find examples of superior Cel7A enzymes in nature, we could identify, using computational modeling, the precise structural subsites responsible for these traits and then combine them into a single enzyme chassis. For this work, called “king chimera,” we choose the Cel7A from Penicillium funiculosum as the structural chassis to receive subsites from natural diversity. Subsites from the cbh1 genes from three top performing microbes were identified by structural analysis. Mutants of the P. funiculosum backbone were then generated, purified, and subjected to rigorous rounds of performance testing using programmatically relevant DMR substrates (LTAD). Although many, not all, of the single and double mutations showed improvement compared to the commercial T. reesei enzyme, the engineered, triple chimera showed a 1.55-fold improvement.

TABLE 2
List of the single, double, and triple mutations on the Penicillium
funiculosum backbone along with the performance improvement on
deacetylated mechanically refined pretreated corn stover substrate.

| | | X-fold |
|---|---|---|
| | | Increase @ |
| Cel7A | Mutations | 95h-Tr = 1.0 |
| V101IT198A | 1 + 2 + 3 | 1.55 |
| N195D | | |
| N195DT198A | 2 + 3 | 1.48 |
| V101IT198A | 1 + 2 | 1.30 |
| N195D V101I | 1 + 3 | 1.48 |
| V101I | 1 | 1.23 |
| T198A | 2 | 1.29 |
| N195D | 3 | 1.46 |

TABLE 3
Site mutation candidates

| | | # | Starting | |
|---|---|---|---|---|
| Index | Mutation(s) | Mutations | Protein | Notes |
| 1 | C4G, C72A | 2 | 10-5 | removing disulfide near entrance to make C10-5 |
| | | | | like PƒCel7A; similar to what was done with |
| | | | | TrCel7A in 2018 Nature Comm |
| 2 | N198D | 1 | PƒCel7A | N198 in TrCel7A is N195 in PƒCel7A; so this is |
| | | | | N195D. D is what C10-5 has here. On B2 loop. |
| 3 | T201A | 1 | PƒCel7A | T201 in TrCel7A is T198 in PƒCel7A, so this is |
| | | | | T198A. A is what C10-5 has here. On B2 loop. |
| 4 | V104I | 1 | PƒCel7A | V104 in TrCel7A is V101 in PƒCel7A; so this is |
| | | | | V101I. I is what C10-5 has here. Located at the |
| | | | | base of A1 loop. |
| 5 | N198D, T201A | 2 | PƒCel7A | Combining 2 and 3 from above. N195D/T198A |
| 6 | N198D, V104I | 2 | PƒCel7A | Combining 2 and 4 from above. N195D/V101I |
| 7 | T201A, V104I | 2 | PƒCel7A | Combining 3 and 4 from above. T198A/V101I |
| 8 | N198D, | 3 | PƒCel7A | Combining 2, 3, |
| | T201A, V104I | | | and 4 from above. |
| | | | | N195D/T198A/V101I |
| 9 | G4C, A72C, | multiple | PƒCel7A | Putting TrCel7A 10th |
| | best of 2-8 | | | disulfide into PƒCel7A + |
| | | | | best of 2 through 8 |
| 10 | D52T, best of | 2-5 | TrCel7A | TrCel7A has D52, |
| | 2-8 | | | whereas the other four |
| | | | | have T. B1 loop. |

In an embodiment, a summary of protein production includes strains Cel6A c-terminus his tag, Cel6A-S413P, and C10-C4G-C72A used, respectively in project titled Cel6A endoglucanase, Cel6A point mutant, and the TCF project all of which were performed in culture volumes of 8 L.

TABLE 4
mg of protein loaded per g glucan for each enzyme ratio

| | Cel7A | AST1116 | bG |
|---|---|---|---|
| Low | 1.5 | 13.2 | 0.3 |
| Mid | 7.35 | 7.35 | 0.3 |
| High | 13.2 | 1.5 | 0.3 |

Measuring enhanced cellulase activity in engineered cellobiohydrolases in simple trinary cellulase system will translate to enhanced activity when reconstituted to a complex cellulase mixture.

In a prophetic embodiment, improved Cel7A variants in CBH1-delete cellulase background broth will be tested.

GH7 King Chimera Activity

Methods:

Cel7A-delete complete cellulase broth was produced by growing AST1116, our cbh1-delete strain of T. reesei QM6a, on MA lactose media. After 5 transfers in 100 mL shake flasks, the culture was transferred to 8 L of MA lactose in a 14 L CSTR, grown to produce cellulase, and the clarified broth concentrated by ultrafiltration.

JLT102A (TrCel7A), HC10 (PfCel7A), and the King Chimera were assayed on SOT DMR at several different ratios to the AST1116 delete broth.

The trinary control digestion used 28 mg/g glucan Cel7A variant, 1.98 mg/g E1, and 0.5 mg/g beta glucosidase. Digestions were for 96 hours at 50° C.

Results:

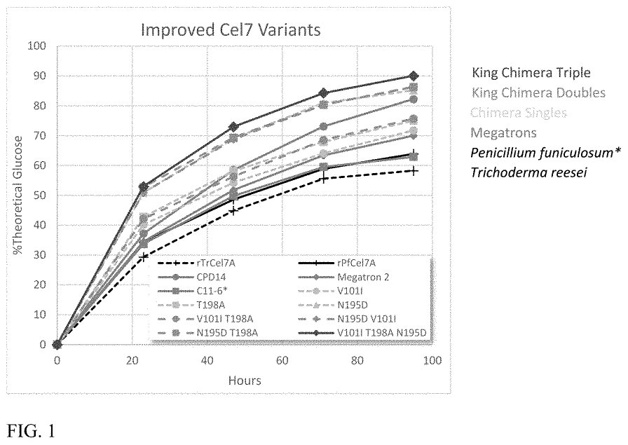

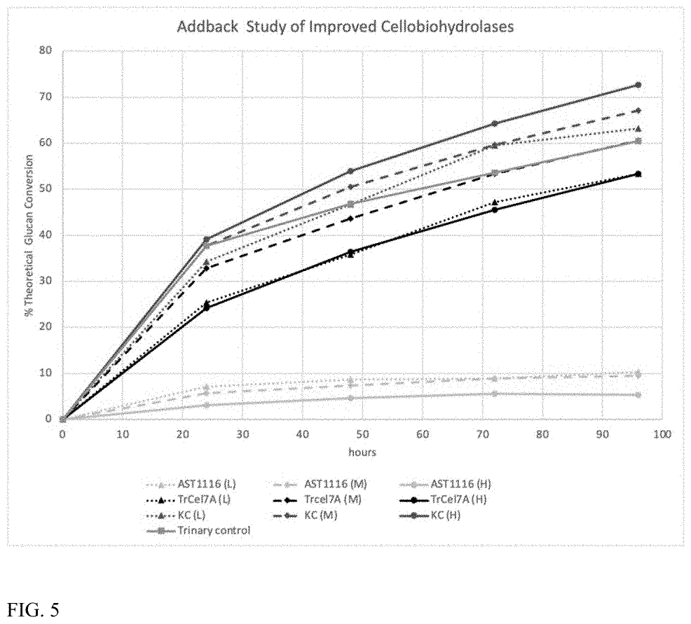

The Cel7A-delete AST1116 broth alone demonstrated very limited conversion of the DMR stover at any loading (yellow lines, FIG. 5), indicating the criticality of Cel7A in the digestion of biomass. The King Chimera Cel7A (red lines, FIGS. 1 and 5) outperformed the native T. reesei Cel7A (black lines, FIG. 1) in the addback study for all three loading ratios tested. It also outperformed the standard trinary cellulase mix loaded at twice the enzyme mass (green line, FIG. 1). This latter observation indicates the clear advantage of a full cellulase suite of activities vs. a simplified mix.

Surprisingly, the ratio of Cel7A to background cellulase did not track as expected. For TrCel7A, the highest activity was seen at the equal loading, with the high TrCel7A and low TrCel7A loadings performing lower. For the King Chimera, the high loading of KCCel7A had the highest conversion of all mixes tested. While it only reached ˜73% conversion, the trajectory of the conversion was still rising. This suggested to us that the native cellulase suite produced by QM6a is significantly inferior to that of a commercially engineered cellulase mix specifically optimized for corn stover. To test this, we removed Cel7A from a commercial cellulase, CTec3, by “purifying” the Cel7A using 5 chromatography steps and then pooling all of the non-Cel7A fractions and unbound flow through materials and concentrating them down using ultrafiltration.

In an embodiment, the King Chimera catalytic domain sequence was added to our synthetic multifunctional “raptor” cellulase. Our previous work demonstrated that building a mini multifunctional cellulase based on the domain structure of CelA from Caldicellulosiruptor bescii improved the combined activity of the two cellulase catalytic domains over their activity when added as individual free enzymes. Our previous raptor consisted of PfCel7A-CBM3-AcCel5A where the cellobiohydrolase catalytic domain from P. funiculosum was connected to a carbohydrate binding module from C. bescii which was connected to an endocellulase catalytic domain from Acidothermus cellulolyticus. We have synthesized a plasmid to replace the PfCel7A domain with the King Chimera domain.

Fermentation and Cell Culture

Three different protein production strains were grown at fed-batch, bench-top fermentation scale, resulting in 30 total liters of T. reesei cell culture. These cultures were protein production runs needed to further investigate cellulase studies on a collaborative project with EEO, the TCF work. One strain was a His-tagged Cel6A expression, used for ease of purification; one strain was a Cel6A endoglucanase variant as a single point mutant; and one was a double mutant in our successful C10 construct.

T. reesei Expression Development

Previous work demonstrated the utility of linking multiple cellulase genes together in a single expression construct using a viral 2A peptide linker system as a means to express multiple genes simultaneously. In an attempt to understand the differential expression levels seen between the 4 genes we linked, we carried out RT-PCR to monitor relative transcription levels of each. For 3 of the 4 genes, the relative transcriptional levels tracked with the protein production levels, however one gene, Cel7B endoglucanase, was highly variable across biological replicates. This variability was confirmed in a second round of RT-PCR, indicating that although transcription and expression levels for Cel7B track together for individual transformants, the Cel7B transcription levels to not track with the other genes in the multi-gene construct.

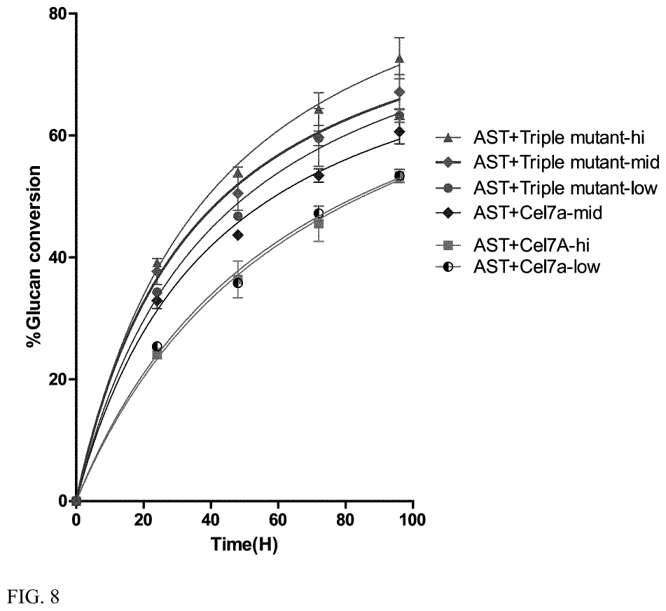

FIG. 8 depicts the results of an addback study comprising Cel7 free “background” broth that was generated from T. reesei, to this was added either wild type Cel7A or the triple mutant. (V101I) (T198A) (N195D). Loadings of the high, mid and low cases are: High: 13.2 mg/g Cel7, 1.5 mg/g AST, Mid: 7.35 mg/g Cel7, 7.35 mg/g AST, and Low: 1.5 mg/g Cel7, 13.5 mg/g AST. As depicted in FIG. 8 In all cases the triple mutant outperforms the Cel7A wild type enzyme in the context of the full industrially relevant T. reesei broth.

The foregoing disclosure has been set forth merely to illustrate the invention and is not intended to be limiting.

We claim:

1. An engineered Cel7A cellobiohydrolase enzyme with greater than 90 percent sequence identity to SEQ ID NO: 1.

2. The engineered Cel7A cellobiohydrolase enzyme of claim 1 comprising a V101I mutation.

3. The engineered Cel7A cellobiohydrolase enzyme of claim 2 wherein the cellobiohydrolase activity is up to about 1.23 times greater than a native Cel7A cellobiohydrolase from Trichoderma reesei.

4. The engineered Cel7A cellobiohydrolase enzyme of claim 1 comprising a N195D and a V101I mutation.

5. The engineered Cel7A cellobiohydrolase enzyme of claim 4 wherein the cellobiohydrolase activity is up to about 1.48 times greater than a native Cel7A cellobiohydrolase from Trichoderma reesei.

6. The engineered Cel7A cellobiohydrolase enzyme of claim 1 comprising a V101I and a T198A mutation.

7. The engineered Cel7A cellobiohydrolase enzyme of claim 6 wherein the cellobiohydrolase activity is up to about 1.30 times greater than a native Cel7A cellobiohydrolase from Trichoderma reesei.

8. The engineered Cel7A cellobiohydrolase enzyme of claim 1 comprising a N195D and a T198A mutation.

9. The engineered Cel7A cellobiohydrolase enzyme of claim 8 wherein the cellobiohydrolase activity is up to about 1.48 times greater than a native Cel7A cellobiohydrolase from Trichoderma reesei.

10. The engineered Cel7A cellobiohydrolase enzyme of claim 1 comprising a V101I, a N195D and a T198A mutation.

11. The engineered Cel7A cellobiohydrolase enzyme of claim 10 wherein the cellobiohydrolase activity is up to about 1.55 times greater than a native Cel7A cellobiohydrolase from Trichoderma reesei.

12. The engineered Cel7A cellobiohydrolase enzyme of claim 10 wherein the cellobiohydrolase activity converts greater than 90% of glucan and xylan monomers of deacetylated mechanically refined pretreated corn stover.

13. An engineered Trichoderma reesei that expresses an engineered Cel7A cellobiohydrolase enzyme with greater than 90 percent sequence identity to SEQ ID NO: 1.

14. The engineered Trichoderma reesei of claim 13 that expresses an engineered Cel7A cellobiohydrolase enzyme comprising a V101I mutation of SEQ ID NO: 1.

15. The engineered Trichoderma reesei of claim 13 that expresses an engineered Cel7A cellobiohydrolase enzyme comprising a N195D and a V101I mutation of SEQ ID NO: 1.

16. The engineered Trichoderma reesei of claim 13 that expresses an engineered Cel7A cellobiohydrolase enzyme comprising a T198A and a V101I mutation of SEQ ID NO: 1.

17. The engineered Trichoderma reesei of claim 13 that expresses an engineered Cel7A cellobiohydrolase enzyme comprising a N195D and a T198A mutation of SEQ ID NO: 1.

18. The engineered Trichoderma reesei of claim 13 that expresses an engineered Cel7A cellobiohydrolase enzyme comprising a N195D, a T198A, and a V101I mutation of SEQ ID NO: 1.

19. A method for degrading cellulose comprising the step of adding an engineered Cel7A cellobiohydrolase enzyme with greater than 90 percent sequence identity to SEQ ID NO: 1 to cellulose.

20. The method of claim 19 wherein the engineered Cel7A cellobiohydrolase enzyme comprises a V101I, a N195D and a T198A mutation of SEQ ID NO: 1.
